(19) United States
(12) Patent Application Publication
(10) Pub. No.: US 2004/0176654 A1
(43) Pub. Date: Sep. 9, 2004

(54) Linear alkylbenzene product and a process for its manufacture

(75) Inventor: Armen Abazajian, Houston, TX (US)

Correspondence Address: JENKENS & GILCHRIST, 1401 MCKINNEY, SUITE 2700, HOUSTON, TX, 77010, US

(73) Assignee: Syntroleum Corporation, Tulsa, OK (US)

(21) Appl. No.: 10/448,663

(22) Filed: May 30, 2003

Related U.S. Application Data
(60) Provisional application No. 60/453,274, filed on Mar. 7, 2003.

(51) Int. Cl. C07C 1/00; C07C 2/00

(52) U.S. Cl. 585/323; 585/1

(57) ABSTRACT

A synthetic olefin/paraffin mixture useful for the production of linear alkybenzene and linear alkybenzene sulfonates. An integrated Fischer-Tropsch process to manufacture linear alkylbenzene and linear alkylbenzene sulfonates.

### CROSS REFERENCE TO RELATED APPLICATIONS

This application claims priority to U.S. provisional application Serial No. 60/453,274, filed on Mar. 7, 2003.

### FEDERALLY SPONSORED RESEARCH

Not applicable.

### REFERENCE TO MICROFICHE APPENDIX

Not applicable.

### FIELD OF THE INVENTION

The invention relates generally to a process for producing alkylbenzenes, and more specifically, modified linear alkylbenzenes. The invention further relates to an integrated Fischer-Tropsch process for the production of the modified linear alkylbenzenes, and feedstock therefor.

### BACKGROUND OF THE INVENTION

Linear alkylbenzene (“LAB”) is an intermediate used to produce linear alkylbenzene sulfonate (“LAS”). LAS compounds are the most widely used surfactants in the world and are incorporated into detergents and cleaning products. LAS compounds are also used as detergent additives in lubricants and other industrial applications.

LAB feedstock for LAS production is primarily produced from petroleum by-products. High linearity n-paraffins are extracted from kerosene and are used to produce highly linear olefins. The olefins are used to alkylate benzene thereby producing LAB. Methods of alkylating aromatic rings are well known in the art. The resulting linear alkylbenzenes are sulfonated and either overbased for use as detergent additives in lubricants and fuels (heavier sulfonates), or neutralized and used as workhorse detergents in a multiplicity of household and industrial detergent formulations.

Prior to the use of LAS, branched alkylbenzene sulfonates (“BAS”) produced from branched alkylbenzenes (“BAB”), based on propylene tetramer, had been the surfactant of choice. However, BAS did not biodegrade readily, which caused foam accumulation in rivers and lakes. Linear alkylbenzenes, however, are environmentally friendly because of the ability of microorganisms to degrade straight chain hydrocarbons. Since the introduction of LAS use in detergents and surfactants, much effort has been expended to improve the paraffin feedstocks used to produce LAS as well as the alkylation process itself in order to achieve highly linear alkylbenzenes. Linear purities of alkyl feedstocks for LAB production typically range from between about 92% and 98%, depending upon the specifications of the end product. The process of extracting such highly linear alkyls from petroleum products, however, involves the use of shape-selective molecular sieves and is, therefore, quite expensive. As a consequence, the cost of products into which such highly linear alkyls are incorporated is increased. Similarly, efforts to improve the alkylation process have further driven up the costs of the LAB and concomitant products.

There remains a need therefore, for a more economical detergent grade linear alkylbenzene and a process for producing such linear alkylbenzene.

### SUMMARY OF THE INVENTION

The invention meets these and other needs, as shall become apparent from the description below, by providing a linear alkylbenze produced from an olefin/paraffin mixture wherein the olefin content is between about 5% and 30%, and containing mono-methyl-branched substituent groups with predominantly terminal branches.

Also provided is an olefin/paraffin mixture, derived from a Fischer-Tropsch synthesis, useful as a feedstock to make the modified alkylbenzene.

An integrated Fischer-Tropsch process to make linear alkylbenzene and linear alkylbenzene sulfonate wherein the olefin/paraffin mixture and/or benzene used in the alkylation process are derived from the Fischer-Tropsch synthesis is also provided.

### BRIEF DESCRIPTION OF THE DRAWINGS

FIG. 1 is a schematic of an embodiment of the integrated Fischer-Tropsch process.

### DESCRIPTION OF EMBODIMENTS OF THE INVENTION

The integrated Fischer-Tropsch process includes processing of synthesis gas to produce a hydrocarbon stream via a Fischer-Tropsch synthesis, recovery, fractionation and separation of the Fischer-Tropsch product into naphtha, light Fischer-Tropsch liquid (“LFTL”) and heavy Fischer-Tropsch liquid (“HFTL”) streams. Each of these streams may be further processed to obtain feedstock material for production of alkylbenzene. Such further processing includes dehydration of the LFTL, hydrocracking of the HFTL, hydrotreatment of the naphtha, and reforming the naphtha from the hydroprocessed HFTL fraction and naphtha recovered from the LFTL. A wide variety of Fischer-Tropsch reaction processes are known in which reaction conditions, catalysts, and reactor configurations vary. The integrated Fischer-Tropsch process of the invention may be used with any such reaction conditions, catalysts, and reactor configurations. For the purposes of the description below, one known Fischer-Tropsch synthesis is described. Other variations of Fischer-Tropsch synthesis are described, inter alia, in U.S. Pat. Nos. 4,973,453; 6,172,124; 6,169,120; 6,130,259, 6,239,184, 6,277,894, 6,344,491; the disclosures of which are all incorporated herein by reference.

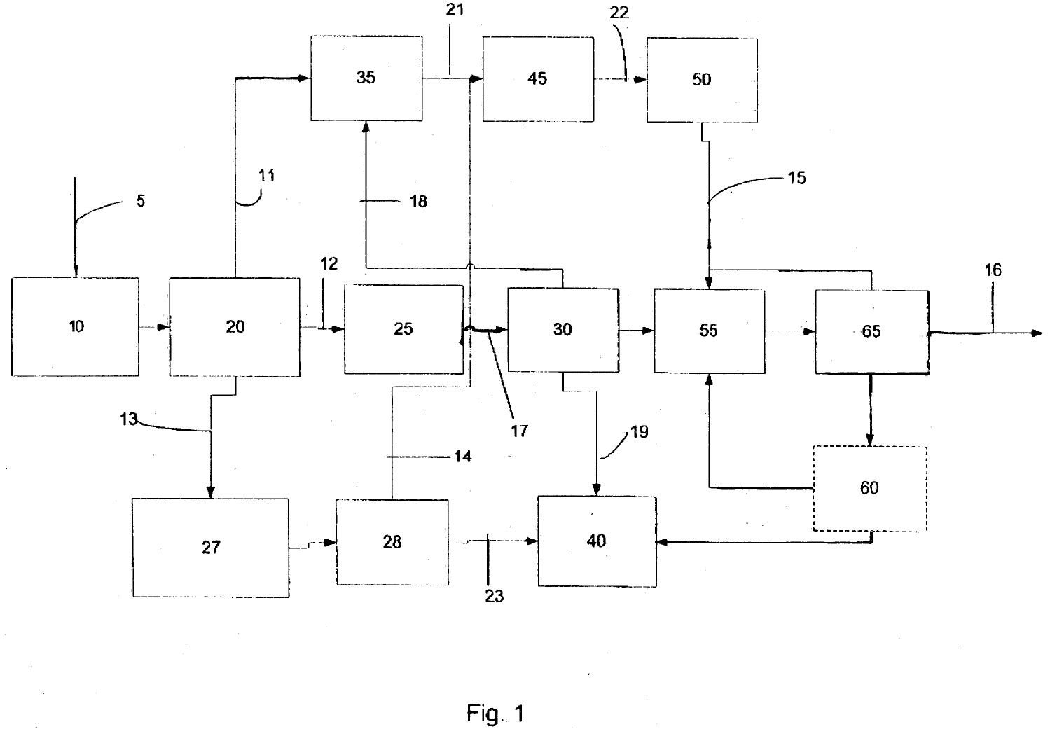

Referring first to FIG. 1, a first embodiment of the integrated Fischer-Tropsch process is described. Synthesis gas is delivered through line 5 to a Fischer-Tropsch synthesis unit 10, which includes a Fischer-Tropsch reactor (FTR) containing a Fischer-Tropsch catalyst.

A detailed description of reforming of natural gas or other hydrocarbonaceous feedstocks to synthesis gas in the presence of air and/or other nitrogen containing gasses is found in co-pending, commonly-owned US patent entitled “Integrated Fischer-Tropsch Process with Enhanced Oxygenates Processing Capability” and listing Armen Abazajian as an inventor, the disclosure of which is incorporated in its entirety herein by reference. This co-pending application also discloses in detail a process for converting the synthesis gas, which contains between about 10 and 60 wt % nitrogen, to hydrocarbons via a Fischer-Tropsch synthesis.

Numerous Fischer-Tropsch catalysts may be used in carrying out the reaction. These include cobalt, iron, ruthenium as well as other Group VIIIB transition metals or combinations of such metals, to prepare both saturated and unsaturated hydrocarbons. The Fischer-Tropsch catalyst may include a support, such as a metal-oxide support, including silica, alumina, silica-alumina or titanium oxides. For example, a Co catalyst on transition alumina with a surface area of approximately 100-200 m²/g may be used in the form of substantially spherical particles of about 50-150 μm in diameter. The Co concentration on the support may also be 15-30%. Certain catalyst promoters and stabilizers may be used. The stabilizers include Group IIA or Group IIIB (others) metals, while the promoters may include elements from Group VIII or Group VIIB (others). The Fischer-Tropsch catalyst and reaction conditions may be selected to be optimal for desired reaction products, such as for hydrocarbons of certain chain lengths or number of carbon atoms. Any of the following reactor configurations may be employed for Fischer-Tropsch synthesis: fixed bed, slurry bed reactor, ebullating bed, fluidizing bed, or continuously stirred tank reactor (CSTR). The FTR may be operated at a pressure of about 100 to about 500 psia and a temperature of about 200° F. to about 500° F. The reactor gas hourly space velocity (“GHSV”) may be from about 1000 to about 8000 hr-1. Syngas useful in producing a Fischer-Tropsch product useful in the invention may contain gaseous hydrocarbons, hydrogen, carbon monoxide and nitrogen with H₂/CO ratios from about 1.8 to about 2.4. The hydrocarbon products derived from the Fischer-Tropsch reaction may range from methane (CH₄) to high molecular weight paraffinic waxes containing more than 100 carbon atoms.

Referring still to FIG. 1, the Fischer-Tropsch LFTL and HFTL produced in synthesis unit 10 are fractionated in a fractionating unit 20 into a naphtha stream through line 11, remaining LFTL through line 12, and HFTL through line 13. The naphtha stream is comprised primarily of C₆-C₁₀ hydrocarbons; the LFTL is comprised primarily of C₁₀-C₂₀ hydrocarbons; and the HFTL is comprised primarily of C₁₈₊ hydrocarbons. Each of these cuts of the Fischer-Tropsch product may also contain small amounts of oxygenates, such as alcohols.

All or part of LFTL fraction, is dehydrated in dehydration unit 25. In the integrated Fischer-Tropsch process, primary and internal alcohols present in the LFTL are dehydrated in the presence of an alumina catalyst to yield corresponding olefins, according to the following reaction:

R—CH₂—CH₂—OH→R—CH═CH₂+H₂O (1)

wherein R is an alkyl group and R—CH₂—CH₂—OH is an alcohol having a boiling point such that it is distilled as part of the LFTL. Thus, the olefin content is enhanced by dehydration of the alcohol component of the oxygenates produced in the Fischer-Tropsch synthesis. A more detailed description of the dehydration process is described in co-pending commonly-owned application entitled “Integrated Fischer-Tropsch Process With Improved Oxygenate Processing Capability” with Armen Abazajian as the first named inventor, the disclosure of which is incorporated herein by reference.

After dehydration, the dehydrated LFTL fraction in line 17 may optionally be further distilled to remove remaining C₁₀₋ and C₁₄₊ component hydrocarbons. This second fractionation, indicated as fractionation 30, may be customized to fit the specifications of particular end products but is not a necessary part of the invention. The C₁₀₋ fraction may be passed to hydrotreater 35 through line 18 and the C₁₄₊ fraction may be passed into storage 40 through line 19 for other use.

The LFTL fraction prior to dehydration will contain a mixture of between about 2% and about 20% alpha-olefins, between about 2% and about 15% internal olefins, between about 2% and about 20% primary alcohols, and between about 2% and about 15% of mono-methyl branched paraffins. Unless otherwise noted, all percentages herein are by weight. Following dehydration the LFTL fraction may contain from between about 5% and about 55% olefins. That is, the dehydration step of the integrated Fischer-Tropsch process can significantly increase the olefin content of the LFTL.

The naphtha stream recovered in line 11 is hydrotreated in hydrotreater 35 and the hydrotreated product in line 21 is passed into catalytic reformer 45 where it is converted into aromatics which are recovered through line 22. Reforming of naphtha into aromatics is well known in the art, and a number of commercially licensed processes are available, such as those processes known by the following marks CCR PLATFORMING™ (UOP), AROMIZING (IFP), and RHENIFORMING® (Chevron). Such processes are disclosed inter alia in U.S. Pat. Nos. 4,347,394; 4,814,533; 6,495,487; and 6,295,219, the disclosures of which are incorporated herein by reference.

The HFTL may be passed to hydrocracker 27 and the hydrocraker product fractionated in fractionating unit 28 to recover a hydrocracked C₆-C₁₀ fraction through line 14 through which it is passed to reformer 45. Heavier portions of the hydrocracked HFTL may be passed to storage 40 through line 23.

The aromatic mixture produced in reformer 45 generally contains a mixture of benzene, toluene, xylene, and other aromatic compounds. Benzene may be extracted from this mixture by any of several known methods including, for example, those marketed under the following marks, MORPHYLANE® (Uhde), GT-BTX® (GTC Technology Inc.) an UDEX™ (UOP). Examples of such processes are disclosed in U.S. Pat. Nos. 5,792,338; 5,073,669; 5,139,651; 4,188,282; and 4,347,394, the disclosures of which are incorporated herein by reference. Alternatively, the mixture may be enriched in benzene prior to extraction. Separation of benzene from the aromatic mixture is illustrated in FIG. 1 at 50. The recovered benzene is then fed into alkylation unit 55 via line 15.

The dehydrated LFTL is reacted with benzene in alkylation unit 55 to produce the linear alkylbenzene. Methods of alkylating benzene are well known, a number of which are described in U.S. Pat. Nos. 5,276,231; 6,392,109; 6,187,981; and 3,681,442, the disclosures of which are incorporated herein by reference. Typically, a large excess, approximately 10:1, of benzene is used in the alkylation process. The olefins present in the dehydrated LFTL react with the benzene to form the linear alkylbenzene product. The paraffin content of the dehydrated LFTL, which comprises from between about 70% and 95% of the alkyl feed, is inert and does not react with the benzene. The product of alkylation unit 55 may be distilled 65 to separate unreacted benzene and paraffins away from the alkylbenzene product. Unreacted benzene may also be separated from the unreacted paraffins and recycled to the alkylation unit 55. Unreacted paraffins from alkylation unit 55 may be blended into a diesel or kerosene product. LAB product is recovered through line 16.

In an alternative embodiment of the invention, the unreacted paraffins from alkylation unit 50 are further processed to be used as feedstock for the alkylation process. More specifically, the unreacted paraffins may be isomerically distilled to recover a paraffinic product having a high purity of linearity and further wherein a portion of the isoparaffinic content is terminal monomethyl branched. Such a process is described in detail in co-pending, commonly-owned U.S. application entitled “Hydrocarbon Products and Methods of Preparing Hydrocarbon Products” Provisional Application No. 60/448,586, naming Armen Abazajian as inventor, the disclosure of which is incorporated herein by reference. The optional isomeric distillation and dehydrogenation processes are shown in FIG. 1 at 60. A 97+% purity n-paraffin stream and an isoparaffinic stream having between about 20% and about 70% terminal monomethyl branches may be isomerically distilled from the unreacted paraffins. The 97+% n-paraffin and/or terminal monomethyl-branched paraffin stream may be dehydrogenated to obtain corresponding olefins. The dehydrogenation may be accomplished using any of a number of known methods. The dehydrogenation product may then be used as alkylation feed to alkylation unit 55. Use of the terminal monomethyl isoparaffins results in production of a modified-linear alkylbenzene (“MLAB”) as described in the previously incorporated application entitled “Hydrocarbon Products and Methods of Preparing Hydrocarbon Products.”

In yet another embodiment, a 94+% purity n-paraffin stream containing slightly higher amounts of isoparaffins may be isomerically distilled from the recovered alkyls. This stream is then dehydrogenated to produce an olefin mixture containing at least about 94% unbranched olefins and between about 0% and 5% branched olefins of which between about 20% and 70% are terminal monomethyl branched. This mixture of olefins may be recycled into alkylation unit 55 and reacted with benzene to form an LAB product. Because terminal monomethyl branched linear alkylbenzene sulfonates have been shown to biodegrade equivalently to the linear alkylbenzene sulfonates, the inclusion of terminal methyl branched paraffins will not impair the biodegradability of the final product.

The ratio of terminal monomethyl to internal monomethyl branching in the Fischer-Tropsch produced paraffins may range from 1:1.5, 1:1, 1.5:1, 2:1, or greater. Because of the random nature and wide distribution of branched moieties in mineral-based paraffin streams, the branched paraffins produced in the Fischer-Tropsch synthesis uniformly contain greater amounts of terminal monomethyl branching as well as higher terminal to internal monomethyl branching.

The linear alkylbenzene, which may contain some methyl-branched alkylbenzene, may then be sulfonated using any of a number of known methods to produce LAS or modified-linear alkylbenzene sulfonates (“MLAS”), respectively. Such methods include those discussed in Detergent Manufacture Including Zeolite Builders and Other New Materials, by Marshall Sittig, Noyes Data Corporation, Park Ridge, N.J., 1979 and in Volume 56 of “Surfactant Science” series, Marcel Dekker, Inc., New York, N.Y., 1996, the disclosures of which are incorporated herein by reference.

Because of the predominantly terminal nature of the monomethyl branching in the paraffins produced in the Fischer-Tropsch reaction, the LAS from LAB, which may include some MLAS from MLAB, are readily biodegraded achieving biodegradation similarly to the LAS products produced using highly linear mineral-based paraffins. The LAS produced using the 94+% n-paraffin stream from the recovered alkyls exhibits biodegradability equal to or greater than commercially available LAS derived from mineral-based paraffins. Indeed, because any remaining branched paraffins in the 97+% or 94+% stream are primarily terminal monomethyl branched, the biodegradability of the LAS produced from the process of the invention may exhibit greater biodegradability than mineral-based LAS.

In yet another embodiment of the invention, natural gas liquid is fractionated to recover a nominal C₆-C₁₀ fraction. Such C₆-C₁₀ fraction may then be processed through a catalytic reformer to produce aromatics. The aromatic product of the reformer is then treated as described above to separate out benzene.

### EXAMPLE 1

A pilot installation consisting of two distillation columns was used to produce C₆₋₁₀ naphtha, C₁₀₋₁₃ stream, and C₁₃₋₂₀₊ stream. The columns were fed approximately 3400 g/hr of liquid Fischer-Tropsch oil. The Fischer-Tropsch oil had approximately the following composition:

| Carbon # | % by wt. |
|---|---|
| 4 | <0.1 |
| 5 | 0.01 |
| 6 | 0.3 |
| 7 | 1.0 |
| 8 | 2.9 |
| 9 | 5.9 |
| 10 | 8.1 |
| 11 | 9.2 |
| 12 | 9.5 |
| 13 | 9.2 |
| 14 | 8.4 |
| 15 | 7.9 |
| 16 | 7.1 |
| 17 | 6.2 |
| 18 | 5.4 |
| 19 | 4.6 |
| 20 | 3.7 |
| 21 | 3.0 |
| 22 | 2.3 |
| 23 | 1.7 |
| 24 | 1.2 |
| 25+ | 2.6 |
| Total | 100.00 |

The Fischer-Tropsch oil was fed into the first column and C₁₃ and lighter materials were distilled overhead. The column conditions were: 10 psig pressure, 480° F. feed preheat temperature, 407° F. overhead temperature, 582° F. bottoms temperature. The first column had approximately 98 inches of Sulzer Mellapack 750Y packing. The overheads of the first column were fed into the second column operating at 12 psig pressure, 370° F. overhead temperature and 437° F. bottoms temperature. The second column was packed with 28 inches of Sulzer EX packing. The bottoms of the second column constituted a nominal C₁₀₋₁₃ stream. The bottoms of the first column constituted a nominal C₁₃₋₂₀₊ stream. The compositions of the C₁₀₋₁₃ stream (Feed A) and the C₁₃₋₂₀₊ stream (Feed B) are shown in Tables 1 and 2, respectively.

TABLE 1

| Total n-paraffins, isoparaffins, | |
|---|---|
| olefins and alcohols | Mass % |
| C7− | 0.02 |
| C8 | 0.25 |
| C9 | 1.29 |
| C10 | 9.83 |
| C11 | 33.51 |
| C12 | 43.04 |
| C13 | 11.47 |
| C14 | 0.49 |
| TOTAL C15+ | 0.10 |
| | 100.00 |

TABLE 2

| Total n-paraffins, isoparaffins, olefins | |
|---|---|
| and alcohols | Mass % |
| C11−: | 0.97 |
| C12: | 1.77 |
| C13: | 11.43 |
| C14: | 13.68 |
| C15: | 12.35 |
| C16: | 10.96 |
| C17: | 9.06 |
| C18: | 7.84 |
| C19: | 6.79 |
| C20: | 7.04 |
| C21: | 5.66 |
| C22: | 4.63 |
| C23+: | 7.83 |
| | 100.00 |

### EXAMPLE 2

30 cc/hr of a Feed A from Example 1 was fed via a syringe pump and mixed with 20 cc/min of nitrogen gas. The gas/liquid mixture was introduced upflow into a vessel packed with stainless steel mesh saddles, where the liquid was vaporized and superheated to reaction temperature of 560° F. The vaporized feed was fed upflow into a reactor packed with ⅛ Alcoa S-400 alumina catalyst and suspended in a heated sandbath. The sandbath was maintained at the reaction temperature and ebulated by air. Reactor LHSV was maintained at about 0.26 hr⁻¹. The reactor outlet was condensed and Product A and water by-product was collected in a product accumulator. System pressure was maintained by controlling the product accumulator overhead pressure at 50 psig. A water layer was drained and Product A was analyzed in a HP 5890 Series II GC with a 60 m RTX1 capillary column with a 0.32 mm bore and 3-micron film thickness. The compositions of Feed A and Product A are reported in Table 3. Product A was also analyzed on a ¹H NMR 300 MHz JOEL analyzer, which analysis confirmed the complete absence of alcohols.

### EXAMPLE 3

15 cc/hr of Feed A from Example 1 was processed in a benchscale process similar to that described in Example 2. Feed A was vaporized and superheated to 650° F. Reactor LHSV was approximately 0.13 hr⁻¹. Composition of Product B from this example is reported in Table 3. ¹H NMR analysis confirmed the absence of alcohols in Product B.

TABLE 3

| Sample Reference Number | | Feed | Product A | Product B |
|---|---|---|---|---|
| TOTAL | | | | |
| N-PARAFFIN | mass % | 80.64 | 80.23 | 79.90 |
| ALPHA OLEFIN | mass % | 4.43 | 8.20 | 7.96 |
| INTERNAL OLEFIN | mass % | 3.04 | 3.37 | 3.91 |
| BRANCHED PARAFFIN | mass % | 8.21 | 8.19 | 8.22 |
| ALCOHOL | mass % | 3.68 | 0.00 | 0.00 |
| | mass % | 100.00 | 100.00 | 100.00 |

### EXAMPLE 4

Feed A from Example 1 was spiked with approximately 5% of hexanol. The hexanol spiked feed is referred to a Feed Aₕₑₓ. Feed Aₕₑₓ was fed at 15 cc/min into a benchscale process as described in Example 3. Nitrogen feed was maintained at 10 cc/min. Composition of Product C from this example is reported in Table 4. ¹H NMR analysis confirmed absence of alcohols in the Product C.

TABLE 4

| | | Feed Aₕₑₓ | Product C |
|---|---|---|---|
| TOTAL | | | |
| N-PARAFFIN | mass % | 75.12 | 75.14 |
| ALPHA OLEFIN | mass % | 4.15 | 10.75 |
| INTERNAL OLEFIN | mass % | 3.03 | 4.47 |
| BRANCHED PARAFFIN | mass % | 9.67 | 9.64 |
| ALCOHOL | mass % | 8.03 | 0.00 |
| | mass % | 100.00 | 100.00 |

### EXAMPLE 5

An LFTL feed, substantially having the composition shown in Table 1, was hydrotreated at reactor conditions of 800 psig and a temperature of 550° to about 590° F. The resulting hydrotreated stream was distilled under conditions as described in Example 1 forming a hydrotreated analog of the C₁₀-C₁₃ stream from Example 1 (Feed A). This hydrotreated stream was analyzed on a Hewlett Packard Series II gas chromatograph with 60 m RTX 1 column with 0.32 mm diameter and 3 micron film thickness. The isomer content of this hydrotreated stream product is shown in Table 6.

TABLE 6

| Component | Wt. % |
|---|---|
| nC9 - | 0.02 |
| 2- and 3-monomethyl C10 | 0.20 |
| 4- and higher - monomethyl C10 | 0.03 |
| nC10 | 22.22 |
| 2- and 3-monomethyl C11 | 1.19 |
| 4- and higher - monomethyl C11 | 0.42 |
| nC11 | 27.93 |
| 2- and 3-monomethyl C12 | 1.09 |
| 4- and higher - monomethyl C12 | 0.50 |
| nC12 | 24.96 |
| 2- and 3-monomethyl C13 | 0.92 |
| 4- and higher - monomethyl C13 | 0.48 |
| nC13 | 18.99 |
| 2- and 3-monomethyl C14 | 0.11 |
| 4- and higher - monomethyl C14 | 0.13 |
| nC14 | 0.41 |
| Total normal hydrocarbon | 94.54 |
| Total 2- and 3- monomethyl | 3.51 |
| substituted hydrocarbons | |
| Total 4- and higher - | 1.55 |
| monomethyl substituted | |
| hydrocarbons | |
| Total monomethyl hydrocarbons | 99.61 |
| Others | 0.39 |

While the foregoing describe preferred embodiments of the invention, it is apparent that a number of changes and variations are within the scope and spirit of the invention.

What is claimed is:

1. A process to produce an alkylbenzenes comprising the steps of:
(a) producing a synthetic crude by Fischer-Tropsch reaction of synthesis gas;
(b) fractionating the synthetic crude at least into a naphtha stream, a light Fischer-Tropsch liquid, and a heavy Fischer-Tropsch liquid;
(c) fractionating light Fischer-Tropsch liquid into at least a naphtha cut and a remaining light Fischer-Tropsch liquid stream;
(d) reacting at least a part of the remaining light Fischer-Tropsch liquid stream over an alumina catalyst to dehydrate alcohols in the remaining light Fischer-Tropsch liquid stream to corresponding alpha- and internal-olefins and forming a dehydrated light Fischer-Tropsch liquid;
(e) reacting the dehydrated light Fischer-Tropsch liquid with benzene to produce an alkylbenzene.

2. The process of claim 1 further comprising the step of:
(f) separating the alkylbenzene from unreacted hydrocarbons and benzene.

3. The process of claim 1 further comprising the step of:
(g) sulfonating the alkylbenzene.

4. The process of claim 1 wherein all or part of the benzene is produced from a process comprising the steps of:
(1) catalytically reforming the naphtha to form a mixture of aromatics; and
(2) processing the product of step (1) and recovering a benzene fraction.

5. The process of claim 1 wherein all or part of the benzene is produced from a process comprising the steps of:
(i) fractionating a natural gas liquid to recover a C₆-C₁₀ fraction;
(ii) catalytically reforming the C₆-C₁₀ fraction to form a mixture of aromatics; and
(iii) processing the product of step (ii) and recovering a benzene fraction.

6. The process of claim 1 wherein the synthesis gas is prepared from a gas comprising methane.

7. The process of claim 6 wherein the synthesis gas is produced by autothermal reformation.

8. The process improvement of claim 7 wherein the synthesis gas comprises between about 10% and about 60% N₂.

9. The process improvement of claim 6 wherein the gas is natural gas.

10. The process improvement of claim 6 wherein the gas is coal gas.

11. The process of claim 1 wherein at least 95 wt % of alcohols present in the light Fischer-Tropsch liquid are converted to olefins in step (d).

12. The process improvement of claim 1 wherein the dehydrated light Fischer-Tropsch liquid contains substantially no alcohols.

13. The process improvement of claim 1 wherein the dehydrated light Fischer-Tropsch liquid contains substantially no oxygenates.

14. The process of claim 1 wherein the dehydration is conducted over a moving bed of alumina catalyst and further comprising continuous catalyst regeneration.

15. The process of claim 14 wherein the moving bed is selected from the group of ebullating beds, slurry bed and a fluidized bed.

16. The process of claim 1 wherein the alumina catalyst is silica-alumina.

17. The process of claim 1 further comprising the step of: vaporizing all or part of the light Fischer-Tropsch liquid before step (d).

18. The process of claim 1 further comprising the steps of:
(I) condensing a dehydrated product;
(II) separating aqueous and organic phases of the dehydrated product; between steps (d) and (e).

19. The process of claim 1 wherein the reaction temperature of dehydration in step (c) is between about 400° and about 800° F.

20. The process of claim 1 wherein the alumina catalyst is gamma-alumina.

21. The process of claim 1 wherein the alumina catalyst is passivated alumina.

22. The process of claim 1 wherein the reaction temperature of dehydration in step (c) is between about 500° and about 700° F.

23. The process of claim 1 wherein the reaction temperature of dehydration in step (c) is between about 550° and about 675° F.

24. The process of claim 1 wherein the light Fischer-Tropsch liquid comprises from about 0 wt % to about 95 wt % olefins.

25. The process of claim 1 wherein the light Fischer-Tropsch liquid comprises from about 0.5 to about 40 wt % oxygenates.

26. The process of claim 1 wherein at least 80 wt % of the oxygenates are primary and internal alcohols.

27. The process of claim 2 further comprising the steps of:
(f2) separating and recovering the unreacted benzene from the unreacted paraffins; and
(f3) reacting the recovered benzene with olefins to produce an alkylbenzene.

28. The process of claim 2 further comprising the step of:
(f4) separating and recovering the unreacted paraffins from the unreacted benzene.

29. An alkylbenzene produced by the process of claim 1.

30. An alkylbenzene sulfonate produced by the process of claim 3.

31. An alkylbenzene produced by the process of claim 4.

32. An alkylbenzene produced by the process of claim 5.

33. The process of claim 28 further comprising the step of dehydrogenating the unreacted paraffins.

34. An alkylbenzene produced by the process of claim 28.

35. The process of claim 28 further comprising the step of:
(f5) isomerically distilling the recovered paraffins to produce a stream comprised of 97+% normal paraffins.

36. The process of claim 28 further comprising the step of:
(f6) isomerically distilling the recovered paraffins to produce a stream comprised of isoparaffins wherein between about 20% and about 70% of the isoparaffins are terminal monomethyl branched.

37. The process of claim 36 further comprising the step of dehydrogenating the 97+% normal paraffins to produce linear internal olefins.

38. The process of claim 37 further comprising the step of dehydrogenating the terminal monomethyl paraffin stream to produce terminal monomethyl branched internal olefins.

39. The process of claim 37 further comprising the step of reacting the olefins with benzene to form alkylbenzenes.

40. The process of claim 38 further comprising the step of reacting the branched olefins with benzene to form alkylbenzenes.

41. A feedstock for use in producing a linear alkylbenzene comprising:
at least about 5 wt % olefins;
at least about 5 wt % n-paraffins; and
between about 2 and 80 wt % branched paraffins wherein substantially all of the branch groups are monomethyl and wherein the ratio of terminal monomethyl branching to internal monomethyl branching is at least about 1:1.5.

42. The feedstock of claim 41 wherein the ratio of terminal monomethyl branching to internal monomethyl branching is at least about 1:1.

43. The feedstock of claim 41 wherein the n-paraffins are present in an amount of at least about 20 wt % and wherein the ratio of terminal monomethyl branching to internal monomethyl branching is at least about 1.5:1.

44. The feedstock of claim 41 wherein the n-paraffins are present in an amount of at least about 40 wt % and wherein the ratio of terminal monomethyl branching to internal monomethyl is at least about 2:1.

45. The feedstock of claim 41 wherein the feedstock is a product of a Fischer-Tropsch reaction.

46. The feedstock of claim 45 wherein the Fischer-Tropsch reaction incorporates feed syngas having between about 10% N₂ and about 60% N₂.
